(19) United States
(12) Patent Application Publication
(10) Pub. No.: US 2024/0390659 A1
(43) Pub. Date: Nov. 28, 2024

(54) DEVICE FOR VAGINAL TISSUE TREATMENT

(71) Applicant: REDDRESS LTD., Pardes Hana (IL)

(72) Inventors: Alon KUSHNIR, Givat Ada (IL); Igal KUSHNIR, Pardes Hana (IL)

(73) Assignee: REDDRESS LTD., Pardes Hana (IL)

(21) Appl. No.: 18/691,637

(22) PCT Filed: Sep. 12, 2022
(86) PCT No.: PCT/IL2022/050985

§ 371 Date: Mar. 13, 2024

(30) Foreign Application Priority Data
Sep. 13, 2021 (IL) 286333

(51) Int. Cl. A61M 31/00; A61F 13/20; A61M 1/38

(52) U.S. Cl. CPC A61M 31/00; A61M 1/38; A61F 13/20; A61M 2202/0413; A61M 2210/1475

(57) ABSTRACT

The present invention relates to a device (100) for treating a vaginal lesion, by forming a coagulated blood mass, which is introduced by the device (100) into the vagina. The device (100) comprises an elongated guiding tube (102) having a lumen (104) for receiving whole blood and a plunger (112) having a piston element (114) fitted within the lumen (104) thus defining a blood coagulating space (109) between it and a blood-introducing end (106). The guiding tube (102) is configured for being introduced into the vagina and to discharge a coagulating blood mass formed within the lumen (104), in result of displacement of the displaceable piston (114) element. The coagulated blood mass may be maintained in the vagina by an anchoring element (122) configured to expand within the vagina and maintain there for a desired time and then be retrieved. Methods of treatment are also disclosed.

### TECHNOLOGICAL FIELD

The present disclosure is in the field of medical vaginal treatment.

### BACKGROUND ART

References considered to be relevant as background to the presently disclosed subject matter are listed below:
WO 2019/058373
WO 2019/058375
U.S. Pat. No. 9,180,142

Acknowledgement of the above references herein is not to be inferred as meaning that these are in any way relevant to the patentability of the presently disclosed subject matter.

### GENERAL DESCRIPTION

The present disclosure provides a method and a device for treating or improving condition of a vaginal lesion in a subject. This is performed by forming a coagulated blood mass, i.e. a volumetric clot, designed to be introduced into the vagina and to be maintained there at a desired location. The coagulated blood mass is formed by whole blood being withdrawn from the subject and coagulated ex-vivo, at least up to a certain degree of coagulation, before being introduced into the vagina. The coagulated blood mass may be maintained in the vagina by an anchoring element that is disposed in the vagina between the coagulated blood mass and the vaginal opening. The anchoring element is configured to expand within the vagina and maintain there for a desired time and then be retrieved. The anchoring element may be a tampon or any other suitable element for maintaining the coagulated blood mass within the vagina and optionally absorb vaginal fluids.

Thus, a first aspect of the present disclosure provides a method for treating vaginal lesions in a subject. The method comprising maintaining an elongated coagulated blood mass within the vaginal lumen for a time sufficient for exerting a therapeutic effect. The coagulated blood mass is being prepared from the subject's whole blood and being either (i) a priori coagulated and introduced into said lumen after coagulation or (ii) introduced into the lumen after mixing with a coagulating agent and permit to coagulate in situ.

It is to be noted that any combination of the described embodiments with respect to any aspect of this present disclosure is applicable. In other words, any aspect of the present disclosure can be defined by any combination of the described embodiments.

In some embodiments of the method, the whole blood is permitted to coagulate in a cylindrical vessel before being introduced into said lumen to obtain a desired cylindrical shape of the coagulated blood mass to be suitable for introducing and maintaining within the vagina.

In some embodiments, the method further comprising, prior to said maintaining, withdrawing whole blood from the subject. The withdrawal of the whole blood can be a short period of time before the initiation of the coagulation process of the blood or can be a relatively long time before the initiation of the coagulation process and then it may be preserved in a non-coagulated state by adding anti-coagulating agents into the withdrawn blood.

In some embodiments of the method, the withdrawn whole blood is mixed with an anti-coagulant.

In some embodiments, the method further comprising introducing the subject's whole blood into a lumen of an elongated, hollowed, guiding tube defined between first, blood-introducing/discharging end and second, sealed end to thereby accumulate the whole blood within the lumen of the tube. It is to be noted that any reference to the sealed end should be understood as an end which blood being introduced through the first, blood-introducing end, cannot be discharged therethrough. The sealing for blood flow can be formed at the end or any portion in the lumen between the sealed end and the blood-introducing end.

It is to be noted that the first, blood-introducing/discharging end may be referred to as first end or blood-introducing end for the sake of brevity, though it should be understood as an end for allowing blood introduction and discharge.

The method further includes, either before or after said introducing, mixing said whole blood with one or more coagulating agents and/or anti-anti-coagulating agents and allowing said whole blood to coagulate within the elongated guiding tube to form a coagulated blood mass having a shape imparted by the elongated guiding tube. Namely, maintaining the blood a sufficient time in the tube to solidify to at least some degree that sufficient for the coagulated blood mass to maintain its general shape, e.g., until the blood is no longer in a flowable state. Then, the method further includes inserting said guiding tube into the vagina and discharging the clot at a desired position within the vagina.

In some embodiments of the method, said guiding tube is cylindrical.

In some embodiments of the method, the blood-introducing end comprises a closure allowing introduction of blood therethrough into the tube. In this embodiment, the method may further include removing the closure prior to inserting the guiding tube into the vagina and discharging the coagulated blood mass.

In some embodiments of the method, said closure is formed with a unidirectional valving element for allowing unidirectional flow of blood therethrough into the tube.

In some embodiments of the method, said discharging comprises forcing the coagulated blood mass out of said first end by a piston element inserted through said second end.

In some embodiments, the method further comprising sealing the blood-introducing end following the introduction of the whole blood into the guiding tube to allow sterile coagulation of the blood within the lumen of the tube.

In some embodiments, the method further comprising adjusting the size of the lumen of the tube to define a desired coagulated blood mass.

In some embodiments of the method, said adjustment is performed only along a longitudinal axis defined between the first and second ends, thus, only the length of the coagulated blood mass changes and the diameter remaining the same.

In some embodiments of the method, said adjustment is performed by an axially displaceable piston element defining a blood coagulating space between it and one end of the tube.

In some embodiments of the method, the piston element is at end of a plunger defining a coagulating space between it and one end of the tube. Said discharging the clot at a desired position within the vagina comprises opening the blood-introducing end and axially displacing the plunger toward the opened end.

In some embodiments, the method further comprising introducing an anchoring element into the vagina configured for maintaining the coagulated blood mass in position. The anchoring element can be a tampon or any other medically compatible element for being maintained in the vagina for a selected period of time.

In some embodiments of the method, the anchoring element is positioned between the coagulated blood mass and the vaginal opening.

In some embodiments of the method, prior to introduction of said anchoring element into the vagina, said anchoring element is disposed in the lumen of the elongated, hollowed, guiding tube. The anchoring element is defined between distal and proximal ends, wherein said distal end is sealingly fitted in the lumen to block liquid flow from a blood-coagulating space of the lumen, distally therefrom, towards the sealed end of the tube. Thus, the anchoring element separates the blood-coagulating space from portion of the lumen distally to the blood-introducing end and the sealing of the sealed end may be performed by the anchoring element at a selected portion in the lumen of the tube. The method further comprises discharging the coagulated blood mass into the vagina from the tube followed by the discharging of the anchoring element.

In some embodiments of the method, the anchoring element comprises an expandable member configured to expand upon being discharged from the guiding tube.

In some embodiments of the method, the expandable member comprises absorbing material for allowing absorption of vaginal fluids while anchored in the vagina.

In some embodiments of the method, the anchoring element comprises a retrieving thread extending from the proximal end for facilitating retrieval of the anchoring element. The method further comprises retrieving said anchoring element following a sufficient period of time from its anchoring within the vagina. The retrieval may be carried out after several hours, for example after at least 1, 2, 3, 4, 5, 6, 7, 8, or at least 9 hours following the positioning of the anchoring element within the vagina.

In some embodiments of the method, the distal end is made of or coated by a material that does not adhere to blood, namely a material that does not adhere to the blood, in its coagulated and non-coagulated form.

In some embodiments of the method, the lumen of the guiding tube comprises said one or more coagulating agents and/or anti-anti-coagulating agents, and said mixing is performed by the introduction of the whole blood into the lumen of the guiding tube.

Yet another aspect of the present disclosure provides a device for treating vaginal lesions in a subject. The device comprising an elongated, hollowed, guiding tube defined between first, blood-introducing/discharging end and second, sealed end and having a lumen for receiving whole blood. The device further includes a plunger having a displaceable piston element at one of it ends, said piston element is fitted within the lumen of the guiding tube defining a blood coagulating space between it and the blood-introducing end to thereby define the coagulating blood mass size.

The guiding tube is designed for being introduced into the vagina and configured to discharge a coagulating blood mass, typically through the blood-introducing/discharging end, formed within the lumen, in result of displacement of the displaceable piston element.

In some embodiments of the device, the blood-introducing end comprises a removable closure allowing introduction of blood therethrough into the tube.

In some embodiments of the device, said closure is formed with a unidirectional valving element for allowing unidirectional flow of blood therethrough into the tube.

In some embodiments of the device, said guiding tube comprises one or more coagulating agents and/or anti-anti-coagulating agents for initiating a coagulation process upon receiving whole blood into its lumen.

In some embodiments, the device further comprising an anchoring element that is disposed in the lumen of the guiding tube. The anchoring element is defined between distal and proximal ends, and said distal end is sealingly fitted in the lumen to block liquid flow from a blood-coagulating space of the lumen, defined distally thereto in the direction of the blood-introducing/discharging end, towards the sealed end of the tube.

In some embodiments of the device, the anchoring element comprises an expandable member configured to expand upon being discharged from the guiding tube.

In some embodiments of the device, the expandable member comprises absorbing material for allowing absorption of vaginal fluids while being anchored in the vagina.

In some embodiments of the device, the anchoring element comprises a retrieving thread extending from the proximal end for facilitating retrieval of the anchoring element.

In some embodiments of the device, the distal comprises or coated by a material that does not adhere to blood, namely a material that does not adhere to the blood, in its coagulated and non-coagulated form.

In some embodiments of the device, the plunger comprises a plunger flange to allow placing the device on a surface to thereby allow formation of the clot against gravitation and therefore obtain the desired tubular or cylindrical shape of the coagulating blood mass.

Yet another aspect of the present disclosure provides a kit comprising: (i) the device of any one of the above-described embodiments or combination of embodiments: (ii) blood withdrawing tools for withdrawing whole blood from the subject: and (iii) one or more coagulating agents and/or anti-anti-coagulating agents for coagulating a blood mass from the withdrawn whole blood from the subject.

In some embodiments, the kit or the device is intended for being used in a method for treating vaginal lesion in a subject.

In some embodiments, the kit or the device is intended for being used in a method for forming a blood clot and introduction thereof into the vagina.

In some embodiments, the kit or the device is intended for being used in the method of any one of the above-described embodiments or any combination thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a perspective view of a non-limiting example of an embodiment of the device for forming a coagulated blood mass and introduction thereof into a vagina according to an aspect of the present disclosure.

FIG. 2 is a perspective view of the guiding tube of the device of FIG. 1.

FIG. 3 is a perspective view of the plunger of the device of FIG. 1.

FIG. 4 is a perspective view of the sealing closure of the device of FIG. 1.

FIG. 5 is a perspective view of the base member of the anchoring element of the device of FIG. 1.

FIG. 6 is a perspective view of the anchoring element of the device of FIG. 1.

FIG. 7 is a flow chart of a non-limiting example of the method for treating a vaginal lesion in a subject according to an aspect of the present disclosure.

### DETAILED DESCRIPTION

The following figures are provided to exemplify embodiments and realization of the invention of the present disclosure.

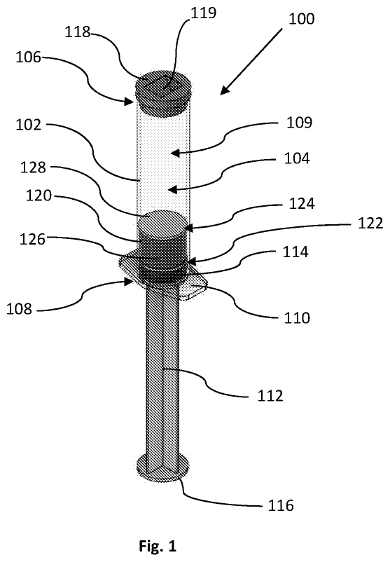

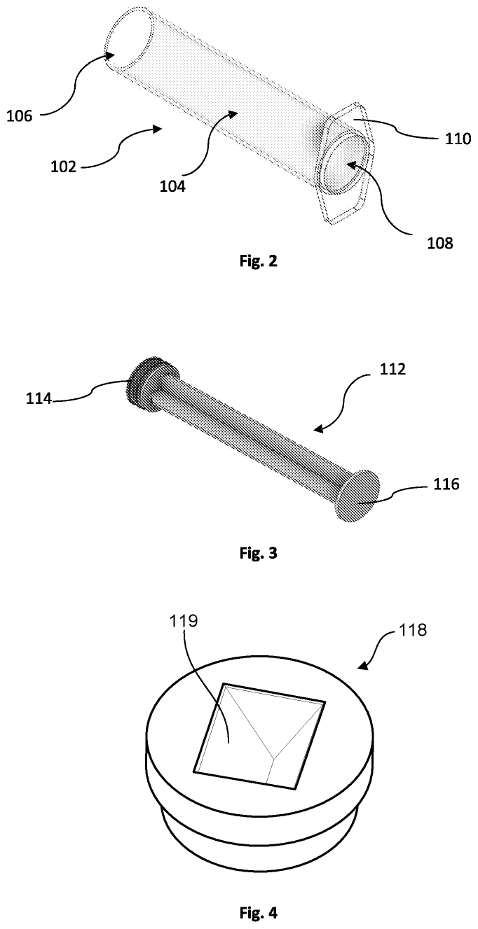

Reference is being made to FIG. 1-6. FIG. 1 is a perspective view of a non-limiting example of a device for forming a coagulated blood mass for introducing into the vagina to thereby treating a vaginal lesion. FIGS. 2-6 show each of the elements of the device alone. It is to be noted that “treating” should be understood as any improvement of the lesion condition. The device 100 comprises an elongated hollowed guiding tube 102 having a lumen 104 and defined between a first, blood-introducing end 106 and a second, plunger-receiving end 108. The lumen 104 includes a blood-coagulating space 109 defined between the blood-introducing end 106 and the first element that seals blood flow from the blood-coagulating space 109 towards the plunger-receiving end 108. The guiding tube 102 is typically cylindrical and can be seen alone in FIG. 2. The guiding tube 102 further includes a guiding tube flange 110 formed at the sealed end 108. Thus, the plunger-receiving end 108 is sealed for liquid flow, namely liquid that is introduced through the blood-introducing end 106 cannot flow and discharge through the plunger-receiving end 108. The sealing can be formed at any location along the lumen 104 between the plunger-receiving end and the blood coagulating space 109.

The device 100 further comprises a plunger 112 having a piston element 114 at one of its ends that is designed to be introduced into the lumen 104 through the plunger-receiving end 108. Typically, the piston element 114 is sealingly fitted within the lumen 104. The plunger 112 comprises a plunger flange 116 that allows to place the device on a surface to permit coagulation of the blood within the lumen against gravitation to receive a desired coagulated blood mass shape. The plunger 112 is shown alone in FIG. 3.

A sealing closure 118 is fitted at the blood-introducing end 106 and is formed with a unidirectional valving arrangement 119 for allowing unidirectional flow of blood into the lumen 104. Thus, the closure allows introduction of whole blood therethrough into the lumen 104 while blocking discharge of blood from the lumen 104 through the blood-receiving end 106. The sealing closure 118 is removable, thus allowing discharge of coagulated blood mass that is formed within the lumen through the first, blood-introducing end 106. The sealing closure 118 is shown alone in FIG. 4.

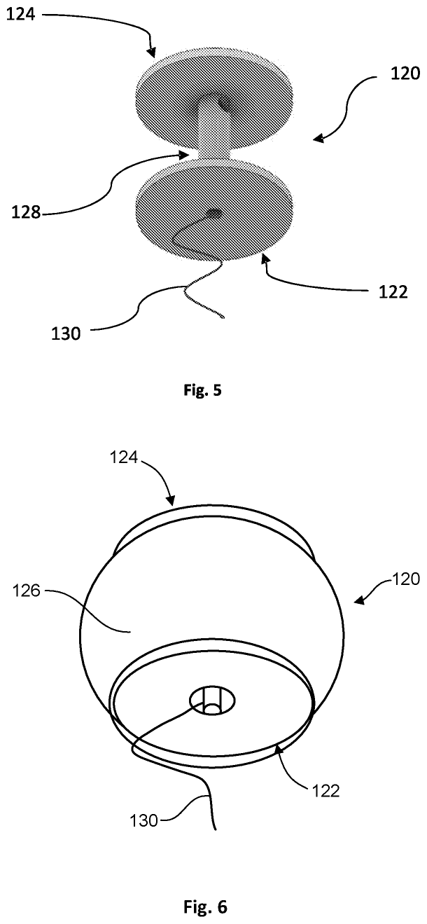

An anchoring element 120 is disposed within the lumen 104 between the piston element 114 and the blood-coagulating space 109. The anchoring element 120 is defined between a proximal end 122 and a distal end 124. Therefore, the blood-coagulating space 109 is defined between the blood-introducing end 106 and the distal end 124 of the anchoring element 120. The anchoring element 120 comprises an expandable member 126 that is configured to expend upon being discharged from the lumen 104. The expandable member 126 is mounted or attached to a base member 128 of the anchoring element 120. The expendable member 126 is formed of an absorbing material to absorb vaginal fluids when it is disposed in the vagina. A retrieving thread 130 extending from the proximal end 122 for allowing retrieving the anchoring element after being disposed in the vagina. It is to be noted that the retrieving thread 130 may be attached either to the proximal end 122 being constituted by the base member 128 or to the expandable member 126. The anchoring element 120 is shown alone in FIGS. 5-6, wherein FIG. 5 shows the base member 128 alone and FIG. 6 shows the base member 128 and the expendable member 126 attached thereto.

In some embodiments, the guiding tube 102 includes one or more coagulating agents and/or anti-anti coagulating agents. Thus, when the whole blood is introduced into the lumen 104 of the guiding tube 102, the coagulation process initiates to form the coagulated blood mass within the lumen 104.

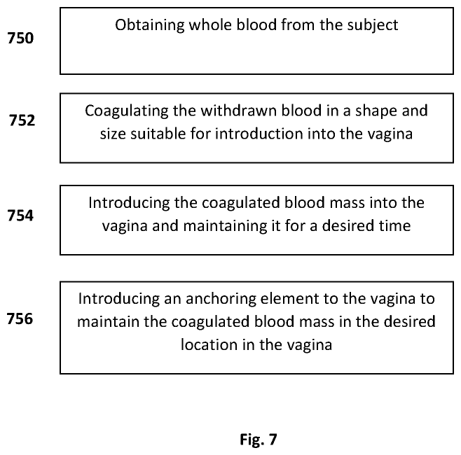

Reference is now being made to FIG. 7, which is a flow chart of a non-limiting example of the method for treating a vaginal lesion in a subject according to an aspect of the present disclosure. The method includes obtaining whole blood of the subject 750. This can be either by withdrawing whole blood from the subject or by obtaining whole blood that was pre-collected from the subject. Then, the method includes coagulating the whole blood, which can be only a portion of the whole blood obtained from the subject, to form a coagulated blood mass having a shape and size suitable for introduction into the vagina of the subject 752. The coagulation of the whole blood can be performed, for example, in an intended tube having a desired shape and size or in any other suitable device that can form the desired coagulated blood mass. When the coagulated blood mass is stable, namely when it is in a solid or at least semi-solid state, the method includes introducing the coagulated blood mass into the vagina and maintaining it for a desired period of time 754 to obtain the desired therapeutic effect to the vaginal lesion. In order to ensure that the coagulated blood mass is maintained in the desired position in the vagina, an anchoring clement is introduced into the vagina 756 and placed between the coagulated blood mass and the vaginal opening.

1. A method for treating vaginal lesions in a subject, comprising:
maintaining an elongated coagulated blood mass within the vaginal lumen for a time sufficient for exerting a therapeutic effect, said mass being prepared from the subject's whole blood and being either (i) a priori coagulated and introduced into said vaginal lumen after coagulation or (ii) introduced into the vaginal lumen after mixing with a coagulating agent and permit to coagulate in situ.

2. The method of claim 1, wherein the whole blood is permitted to coagulate in a cylindrical vessel before being introduced into said lumen.

3. The method of claim 1, comprising, prior to said maintaining, withdrawing whole blood from the subject; wherein the withdrawn whole blood is mixed with an anti-coagulant.

4. (canceled)

5. The method of claim 1, comprising:
introducing the subject's whole blood into a lumen of an elongated guiding tube defined between first, blood-introducing end and second, sealed end;
either before or after said introducing, mixing said whole blood with one or more coagulating agents and/or anti-anti-coagulating agents and allowing said whole blood to coagulate within the elongated guiding tube to form a coagulated blood mass having a shape imparted by the elongated guiding tube; and
inserting said guiding tube into the vagina and discharging the coagulated blood mass at a desired position within the vagina.

6. The method of claim 1, wherein the blood-introducing end comprises a closure allowing introduction of blood therethrough into the tube, the method further comprising, prior to said inserting, removing said closure for allowing discharge of the coagulated blood mass through the first, blood-introducing end; wherein said closure is formed with a unidirectional valving element for allowing unidirectional flow of blood therethrough into the tube.

7. (canceled)

8. The method of claim 5, wherein said discharging comprises forcing the coagulated blood mass out of said first end by a piston element inserted through said second end.

9. The method of claim 5, comprising adjusting the size of the lumen of the tube to define a desired coagulated blood mass.

10. The method of claim 9, wherein said adjustment is performed only along a longitudinal axis defined between the first and second ends; wherein said adjustment is performed by an axially displaceable piston element defining a blood coagulating space between it and one end of the tube wherein
the piston element is at end of a plunger defining a coagulating space between it and one an end of the tube, and wherein
said discharging comprises opening the blood-introducing end and axially displacing the plunger toward the opened end.

11. (canceled)

12. (canceled)

13. The method of claim 1, comprising:
introducing an anchoring element into the vagina configured for maintaining the coagulated blood mass in position.

14. The method of claim 13, wherein
prior to introduction of said anchoring element into the vagina, said anchoring element is disposed in a lumen of an elongated guiding tube defined between first, blood-introducing end and second, sealed end,
said anchoring element is defined between distal and proximal ends, said distal end is sealingly fitted in the lumen to block liquid flow from a blood-coagulating space of the lumen, distally therefrom, towards the sealed end of the tube; and wherein the method comprises discharging the coagulated blood mass into the vagina from the tube followed by the anchoring element; wherein
the anchoring element comprises an expandable member configured to expand upon being discharged from the guiding tube.

15. (canceled)

16. (canceled)

17. The method of claim 13, wherein
the anchoring element comprises a retrieving thread extending from the proximal end for facilitating retrieval of the anchoring element, and wherein
the method further comprises retrieving said anchoring element following a sufficient period of time from its anchoring within the vagina.

18. (canceled)

19. The method of claim 5, wherein the lumen of the guiding tube comprises said one or more coagulating agents and/or anti-anti-coagulating agents, and said mixing is performed by the introduction of the whole blood into the lumen of the guiding tube.

20. A device for treating vaginal lesions in a subject, comprising:
an elongated guiding tube defined between first, blood-introducing end and second, sealed end and having a lumen for receiving whole blood;
a plunger having a displaceable piston element at one of it ends, said piston element is fitted within the lumen of the guiding tube defining a blood coagulating space between it and the blood-introducing end;
wherein said guiding tube is configured for being introduced into the vagina and to discharge a coagulating blood mass formed within the lumen, in result of displacement of the displaceable piston element.

21. The device of claim 20, wherein the blood-introducing end comprises a removable closure allowing introduction of blood therethrough into the tube; wherein said closure is formed with a unidirectional valving element for allowing unidirectional flow of blood therethrough into the tube

22. (canceled)

23. The device of claim 20, wherein said guiding tube comprises one or more coagulating agents and/or anti-anti-coagulating agents for initiating a coagulation process upon receiving whole blood into its lumen.

24. The device of claim 20, comprising an anchoring element disposed in said lumen between the first, blood-introducing end and the second, sealed end, said anchoring element is defined between distal and proximal ends, said distal end is sealingly fitted in the lumen to block liquid flow from a blood-coagulating space of the lumen, distally therefrom, towards the sealed end of the tube.

25. The device of claim 24, wherein the anchoring element comprises an expandable member configured to expand upon being discharged from the guiding tube.

26. The device of claim 25, wherein the expandable member comprises absorbing material for allowing absorption of vaginal fluids while anchored in the vagina.

27. The device of claim 16, wherein the anchoring element comprises a retrieving thread extending from the proximal end for facilitating retrieval of the anchoring element;
wherein the distal end is made of or coated by a material that does not adhere to blood;
wherein the plunger comprises a plunger flange to allow placing the device on a surface.

28. (canceled)

29. (canceled)

30. A kit comprising:
the device of claim 20;
blood withdrawing tools for withdrawing whole blood from the subject; and
one or more coagulating agents and/or anti-anti-coagulating agents for coagulating a blood mass from the withdrawn whole blood from the subject.

31.-33. (canceled)
